(19) United States
(12) Patent Application Publication
(10) Pub. No.: US 2025/0064455 A1
(43) Pub. Date: Feb. 27, 2025

(54) DEVICES AND METHODS FOR VACUUM-ASSISTED TISSUE STABILIZATION

(71) Applicant: BioFluid Technology, Inc., Bryn Mawr, PA (US)

(72) Inventors: Stephen J. KRON, Chicago, IL (US); Reuben E. KRON, Bryn Mawr, PA (US)

(21) Appl. No.: 18/718,867

(22) PCT Filed: Dec. 14, 2022
(86) PCT No.: PCT/US2022/052832

§ 371 Date: Jun. 12, 2024

Related U.S. Application Data
(60) Provisional application No. 63/290,288, filed on Dec. 16, 2021.

(51) Int. Cl. A61B 17/12; A61B 17/00

(52) U.S. Cl. CPC A61B 17/12013; A61B 2017/00561

(57) ABSTRACT

Provided are wound sealer components, comprising: an upper surface, a bottom surface, a vacuum chamber defined between the upper surface and the lower surface, the bottom surface defining at least one vacuum chamber opening in fluid communication with the vacuum chamber, and the vacuum chamber opening being defined between vacuum chamber opening edges of the component; a vacuum tap that places the vacuum chamber into fluid communication with the environment exterior to the vacuum tap, the vacuum tap being adapted for application of vacuum within the vacuum chamber, the at least one vacuum chamber opening being adapted to, upon application of sufficient vacuum from the tap, securably engage with tissue proximate to the bottom surface by drawing at least some of the tissue toward or even into the vacuum chamber so as to stably affix the component to the tissue, and a sealer portion, the sealer portion configured such that application of vacuum to draw tissue toward or even into the vacuum chamber exerts the sealer against the tissue so as to seal an opening present in the tissue.

[Continued on next page]

### CROSS-REFERENCE TO RELATED APPLICATIONS

The present application claims priority to and the benefit of U.S. patent application No. 63/290,288, “Devices And Methods For Vacuum-Assisted Tissue Stabilization” (filed Dec. 16, 2021), the entirety of which application is incorporated herein by reference for any and all purposes.

### TECHNICAL FIELD

The present disclosure relates to the field of tissue stabilizing devices.

### BACKGROUND

Many surgeries—e.g., gynecologic surgeries—entail operating on or nearby to blood vessels, and with this operation comes the attendant risk of “nicking” a blood vessel and inducing unwanted bleeding. In such a situation, the operating surgeon must often attempt to stop the unwanted bleeding—frequently with their own fingers—while awaiting assistance from a vascular surgeon. This approach presents certain risks, as manually stopping blood flow can be imprecise and ineffective, and the time spent waiting for the arrival of the vascular surgeon can present additional risk to the patient. Outside of the operating room and in the field, there are similar issues presented by open wounds—e.g., a puncture wound to the skin or to a blood vessel—that require quick and effective ways to stop unwanted bleeding. Accordingly, there is a long-felt need in the art for improved devices and methods for stabilizing tissue and/or stopping unwanted bleeding.

### SUMMARY

In meeting the described long-felt needs, the present disclosure provides components, comprising: an upper surface, a bottom surface, a vacuum chamber defined between the upper surface and the lower surface, the bottom surface defining at least one vacuum chamber opening in fluid communication with the vacuum chamber, and the vacuum chamber opening being defined between vacuum chamber opening edges of the component: a vacuum tap that places the vacuum chamber into fluid communication with the environment exterior to the vacuum tap, the vacuum tap being adapted for application of vacuum within the vacuum chamber, the at least one vacuum chamber opening being adapted to, upon application of sufficient vacuum from the tap, securably engage with tissue proximate to the bottom surface by drawing at least some of the tissue toward (or even into) the vacuum chamber so as to stably affix the component to the tissue, and one or more of (i) a sealer portion, the sealer portion configured such that application of vacuum to draw tissue toward the vacuum chamber exerts the sealer against the tissue so as to seal an opening present in the tissue, (ii) a projection configured to extend into a wound when the component is engaged with a subject's tissue, the component optionally configured such that application of vacuum to draw tissue toward the vacuum chamber exerts the sealer against the tissue such that the projection extends into a opening of the tissue.

Also provided are methods, the methods comprising contacting a vacuum chamber comprising at least one tissue engagement aperture to a body tissue: and providing sufficient vacuum to the vacuum chamber so as to draw at least some of the body tissue toward (or even into) the vacuum chamber and so as to physically stabilize at least part of the tissue.

Further provided are components, comprising a vacuum chamber, the vacuum chamber comprising a tap adapted for application of vacuum, and the vacuum chamber defining at least one opening formed therein, the at least one opening being adapted to, upon application of sufficient vacuum, securably engage with tissue.

Also provided are methods, comprising contacting a vacuum chamber comprising at least one tissue engagement aperture to a body tissue: and applying sufficient vacuum to the vacuum chamber so as to draw at least some of the body tissue toward (or even into) the vacuum chamber and so as to physically stabilize at least part of the tissue.

Further provided are methods, comprising applying a vacuum via the vacuum tap of a device according to the present disclosure (e.g., any one of Aspects 1-16) so as to sealably affix the component to tissue proximate to the component. As described elsewhere herein, the vacuum can be applied in an automated fashion (e.g., via an automated pump), but can also be applied in a manual fashion, e.g., via a squeeze bulb, pedal, or other manual modality.

Also provided are kits, comprising a first component according to the present disclosure (e.g., according to any one of Aspects 1-16) and a second component according to the present disclosure (e.g., according to any one of Aspects 1-16), the first component and the second component defining different maximum cross-sectional dimensions. As but one example, a kit can comprise components of various sizes such that the kit allows a user to address wounds of varying size. Such a kit could be, for instance, be present in an emergency response vehicle so as to allow the emergency response personnel to address wounds of multiple sizes. The components of such a kit could be, of course, compatible with the same vacuum source so that a single vacuum source could be used to effect reduced pressure within multiple components at the same time.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale or proportion. In the drawings:

FIG. 1 provides a cross-sectional view of a deployed component according to the present disclosure:

FIG. 2 provides a down-looking view of a deployed component according to the present disclosure:

FIG. 3 provides a cross-sectional view of a deployed component according to the present disclosure:

FIG. 4 provides a cross-sectional view of a deployed component according to the present disclosure:

FIG. 5 provides a cross-sectional view of a deployed component according to the present disclosure: and

FIG. 6 provides a cross-sectional view of a deployed component according to the present disclosure.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below; although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value: they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

### FIGURES

The attached figures are illustrative only and do not limit the scope of the present disclosure or the appended claims.

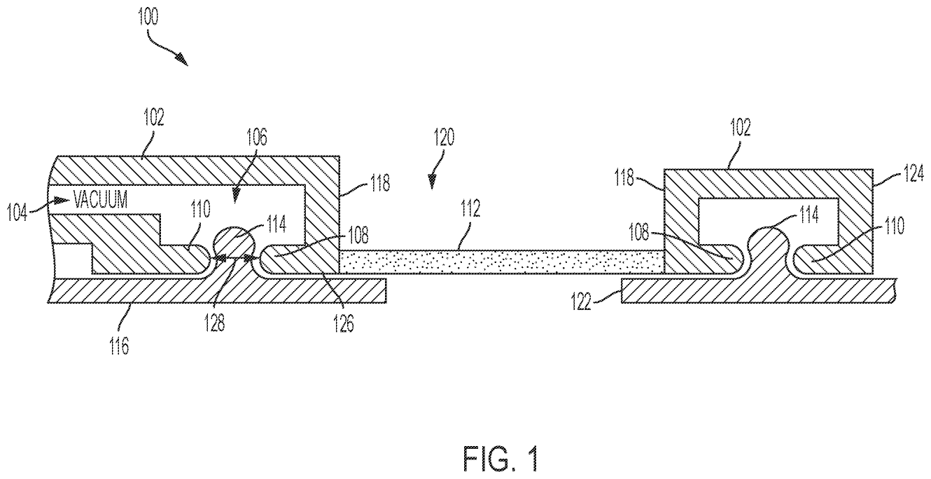

FIG. 1 provides a cutaway view of an exemplary component 100 according to the present disclosure, which figure shows component 100 sealing an opening 122 present in tissue 116. As shown, component 100 can define upper surface 102, inner wall 118, and outer wall 124. As shown, inner wall 118 can enclose component opening 120, which opening can be bounded by inner wall 118 and sealer 112. Component opening 120 can be circular, but can also be square, oval, polygonal, or any other shape. Component openings that are oblong (e.g., oval) in shape can be especially suitable for sealing cuts in blood vessels but this is not a requirement.

Sealer 112 can be an element (e.g., a membrane) that is affixed to component 100, e.g., via heat-sealing, gluing, ultrasonic welding, mechanical joints (e.g., a sandwich joint) or by other techniques known in the art. In some embodiments, sealer 112 is an element separate from the body of component 100, i.e., sealer 112 is not integral with inner wall 118. This is not a requirement, however, as sealer 112 can also be integral with inner wall 118: in some embodiments, component 100 is formed of a single, integral piece of material, e.g., via injection molding, additive manufacturing (e.g., 3-D printing), or even via milling or other subtractive manufacturing.

Sealer 112 can be flush with the bottom surface 126 of component 100, but this too is not a requirement. In some embodiments, sealer 112 can be affixed to bottom surface 126 such that a portion of sealer 112 lies atop bottom surface 126. Sealer 112 can be flat, but this too is not a requirement. As an example, sealer 112 can be shaped such that it is domed outwardly, e.g., such that it domes outwardly away from upper surface 102 of component 100. Without being bound to any particular theory, a domed sealer can extend into an opening in tissue so as to better seal the opening.

Sealer 112 can also include a resealable portion, e.g., a patch or port. Such a resealable potion can be used to administer a treatment (e.g., a clotting agent, a painkiller, and the like) through the sealer to the area beneath the sealer. The sealer can itself have a medicament present thereon, e.g., a clotting agent, an analgesic, and the like, such that the medicament is delivered to tissue that contacts sealer 112.

As shown in FIG. 1, vacuum can be drawn through tap 104 so as to give rise to a reduced or even negative pressure within vacuum chamber 106. This reduced pressure can in turn draw tented tissue 114 through opening 128 defined between vacuum chamber edges 108 and 110 of component 100. Without being bound to any particular theory or embodiment, opening 128 can define a width in the range of from about 0.1 to about 10 cm.

As shown, tented tissue 114 can define an expanded portion, which expanded portion can-without being bound to any particular theory-help to secure tented tissue 114 within vacuum chamber 106. Application of vacuum into vacuum chamber 106 thereby exerts sealer 112 against opening 122, so as to seal said opening.

After tented tissue 114 is secured within vacuum chamber 106, the degree of vacuum exerted through tap 104 can be maintained, but can also be reduced or even removed, although this is not a requirement. Put another way, after a component is attached to tissue, the component can remain in position-while sealing an opening in the tissue-without continued vacuum application. In this way, one can seal a wound and then remove the vacuum source from the sealer so that the patient can be moved without the need to remain attached to the vacuum source. Without being bound to any particular theory or embodiment, the applied vacuum can remain/persist in the component to a degree sufficient to maintain the component in position. The removal of the vacuum source can also provide a better field of view of the wound, as the field of view will not be encumbered by the presence of a connection to the vacuum tap or by the presence of the vacuum source itself. To assist with placement of the device and/or to visualize the wound being sealed, sealing portion 112 can be translucent or even transparent. Likewise, other portions of the component can be translucent or even transparent. The upper surface of the component can be colored (e.g., bright blue, orange) to as to allow for easier visualization of the component to assist with placing the component.

A component may be sized so that it defines a cross-sectional dimension (or even a maximum cross-sectional dimension) of from about 0.01 cm to about 50 cm, or less than 10 cm, less than 5 cm, or even less than 2 cm. For example, the component may have a diameter (defined by the inner diameter of the component opening 120 of the device) of about 3 cm and a height of about 0.5 cm. A component may be wider than it is tall; a component can also be as wide as it is tall, and can also be taller than it is wide. The sealer portion of a component can define a cross-sectional dimension of from about 0.1 to about 5 cm.

By reference to FIG. 1, component opening 120 may define a width that is greater than the height of the component opening. Component opening 120 may define a width that is equal to the height of the component opening. Component 120 can also define a width that is smaller than the height of the component opening.

The component may be configured and sized as to be insertable into a patient by way of an incision or even a so-called keyhole incision. The component may also comprise a deformable material so that at least a portion of the component may be compacted or otherwise folded to ease insertion into a patient and to ease transit within the patient to the desired location. The component may include a deformable material and a rigid material, in some embodiments. A device may be configured so that it expands or otherwise opens once inserted into a patient. In some embodiments, a component may be fabricated of subcomponents that are connected or otherwise assembled with one another once they are placed within a subject.

A component may be constructed so it takes on a curved or other shape within a patient, as needed. The devices may be constructed such that their shape, curvature, or other characteristic may be adjusted while the device is inside of a patient: i.e., be adapted to the operative field in terms of curvature and shape. The component may also be connected to a flexible or rigid conduit or other connection that provides the vacuum to the component once the component has been inserted into the patient. The component may also be connected to another component, device, or connection that holds the component in place. Once the component is positioned, the user may apply a vacuum (suitably less than about 300 mm Hg, 100 mm Hg, 50 mm Hg, 10 mm Hg, or even less than about 5 mm Hg) so as to affix the component to the tissue of interest. The vacuum connection may define a cross-sectional dimension (e.g., diameter, radius) in the range of from about 0.01 mm to about 10 mm or even about 50 mm. The component may also be positioned so as to-when vacuum is applied-seal or otherwise clamp off a blood vessel or other structure. It should be again understood that the opening of the vacuum chamber may be circular, but may also be of another shape, such as square, oblong, elliptical, or other shape.

Tissue 116 can be-but does not have to be-a blood vessel. As shown in FIG. 1, tissue 116 is a blood vessel, which blood vessel defines lumen 118 therein. Other types of tissue besides blood vessels (e.g., skin, cardiac tissue, uterine tissue) can also be sealed with a component according to the present disclosure.

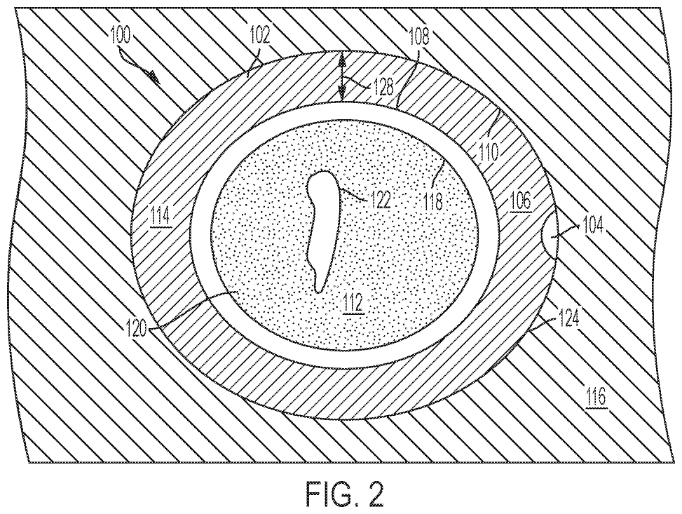

FIG. 2 provides a downward-looking view of a component 100 deployed so as to seal to tissue 116. As shown, component 100 is deployed such that sealer 112 is placed in register with and surrounding opening 122 formed in tissue 116: opening 122 is bounded by inner wall 118, which encloses component opening 120. As shown, vacuum can be applied via vacuum tap 104 so as to draw tented tissue 114 into vacuum chamber 106, between edges 108 and 110 of component 100. Upper surface 102 is also shown, present above opening 128 defined between edges 108 and 110.

As discussed elsewhere herein, sealer 112 can optionally comprise a resealable portion, such as a patch or port. Such a resealable portion can be used to deliver a medicament to opening 122 and/or to tissue 116, including (but not limited to) tissue that is nearby to opening 122. Sealer 112 can be formed of a self-sealing material through which a needle or other instrument can be penetrated, and when the instrument is removed, the material self-seals the opening through which the instrument was extended. Sealer 112 can be a single layer of material, but can also be multiple layers of one or more materials.

It should be understood that although FIG. 2 illustrates an annular vacuum space 106, this is not a requirement, as a component need not have a vacuum space that completely encircles sealer 112 so as to draw an annular ring of tissue 114 toward or even into the vacuum space 106 through an annular opening defined between edge 108 and edge 110. For example, a component can define separate openings in bottom surface 126, through which bottom openings tissue is drawn. The separate openings can be in fluidic isolation from one another, although this is not a requirement. In some embodiments, a single vacuum tap can apply vacuum to two or more openings in bottom surface 126.

As an example, a circular component according to the present disclosure can comprise openings present at the 12 o'clock, 3 o'clock, 6 o'clock, and 9 o'clock positions around the circular component. In this way, tissue drawn toward or even into the openings still exerts the sealer of the component against the opening in the tissue against which the component is positioned. Although in some situations it may be preferable to secure the component to the underlying tissue via a ring of tented skin, this is not a requirement, as individual/discrete portions of tented skin can also secure the sealer against the tissue opening. A sealer can define equally spaced openings around the perimeter of the sealer, e.g., the 12 o'clock, 3 o'clock, 6 o'clock, and 9 o'clock arrangement described above.

An opening through which tissue is drawn can be, as described elsewhere herein, annular in shape. But this is not a requirement, as an opening through which tissue is drawn can be a slot (e.g., a non-circular slot), a hole, ovoid, polygonal, and the like. A component can be configured such that tented tissue openings are positioned directly across the component opening from one another, e.g., positioned at 12 o'clock and 6 o'clock.

The vacuum applied to a component according to the present disclosure can be applied via a machine, e.g., via a powered pump. Vacuum can, however, also be applied via a hand-pumped element, such as a squeeze bulb or other manually-actuated modality. This allows one to perform an in-the-field deployment of the disclosed components without the need for electricity, as electricity is not always available in the field when needed.

The disclosed components can be present as a single component, but can also be present as a kit, e.g., a collection of components of differing sizes and/or shapes. In this way, a user with a kit can select the component having the size and/or shape that is best suited for a particular use. As but one example, a user may prefer to use a relatively small component on a nicked vein, and a relatively larger component on a nicked artery. A user may also decide to use a round-shaped component to seal a circular puncture wound, and use an oval-shaped component to seal a linear nick-shaped wound.

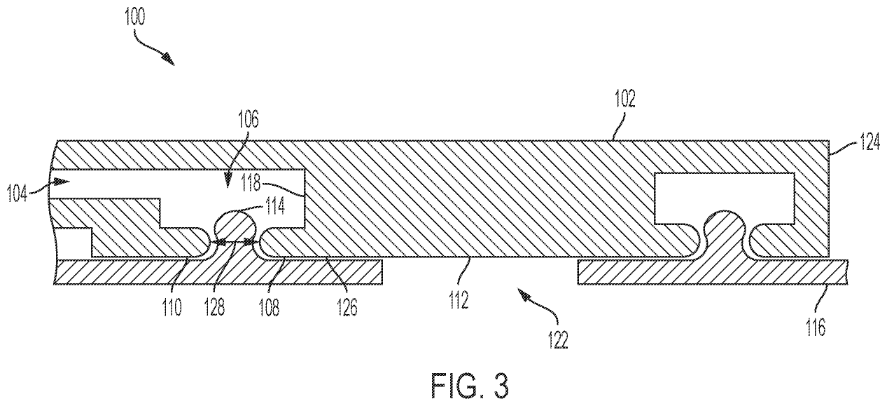

FIG. 3 provides a view of a deployed component 100 according to the present disclosure. As shown, component 100 can define upper surface 102 and bottom surface 126, along with outer or perimeter surface 124. Application of a vacuum through vacuum tap 104 can draw tented tissue 114 through opening 128 and into vacuum chamber 106; as shown, tissue drawn into vacuum chamber 104 can expand, which expansion can—without being bound to any particular theory—maintain component 100 in position on tissue 116. As shown, opening 128 can be defined between edges 108 and 110 of the opening. An edge can define a thickened end (not shown in FIG. 3).

Component 100 can also include sealer 112, which sealer 112 can be a portion of the component: e.g., a surface of a unibody component. Sealer 112 can be exerted against tissue 116 (via application of vacuum in vacuum chamber 104 and drawing tented tissue 114 into vacuum chamber 106), and the exertion of sealer 112 against opening 122 in tissue 116 can act to at least partially seal opening 122.

Sealer 112 can, in some instances, simply be a surface of component 100 and be formed of the same material as the remainder of component 112. Sealer 112 can also comprise a layer or layers of other material (e.g., a flexible material and/or soft material that conforms to tissue 116 and/or opening 122). Sealer 112 can be flush with bottom surface 126, but this is not a requirement, as sealer 112 can (not shown in FIG. 3) can define a domed cross-section so as to better seal opening 122. Component 122 can define a channel, port, or other pathway (not shown) that allows a user to deliver a medicament through sealer 112 to opening 122 and/or tissue 116, including tissue nearby to opening 122.

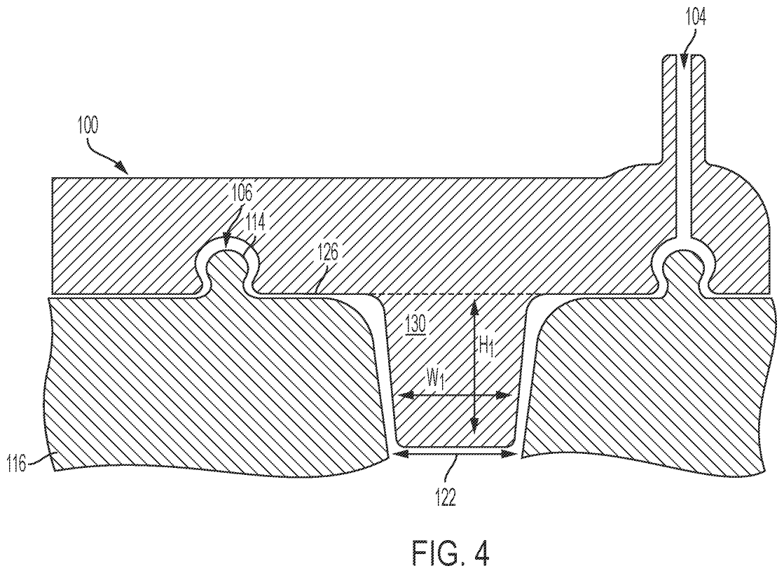

FIG. 4 provides a further alternative embodiment of the disclosed technology. As shown, component 100 can seal a wound (e.g., partially defined by opening 122). As shown, tented tissue 114 (from tissue 116) can be drawn by vacuum applied to tap 104 into vacuum chamber 106; vacuum chamber 106 can extend circumferentially and can be, e.g., toroidal (or partially toroidal) in form. Drawing tented tissue 114 into vacuum chamber 106 can seal component 100 against tissue 116. As shown, component 100 can include projection 130: without being bound to any particular theory, projection 130 can extend into a wound (e.g., a hole-like wound) so as to provide sealing. Projection 130 (or other parts of component 100) can have placed thereon a medicament or other material, e.g., antibiotics and/or hemostatic agents, such as kaolin. Projection 130 can be rigid, but this is not a requirement, as projection 130 can also be flexible. Component 100 can comprise silicone, for example. Component 100 can be impervious, but can also comprise one or more porous regions.

As shown, projection 130 can define a height H1 and a width W1. The width of the projection can vary along the height of the projection. For example, projection 130 can taper. Projection 130 can also be of constant cross-section. The cross section of projection 130 can be circular, but can also be polygonal, ovoid, or other cross-section. H1 can be in the range of from, e.g., about 0.1 cm to about 5 cm. W1 can be in the range of from, e.g., about 0.1 cm to about 5 cm.

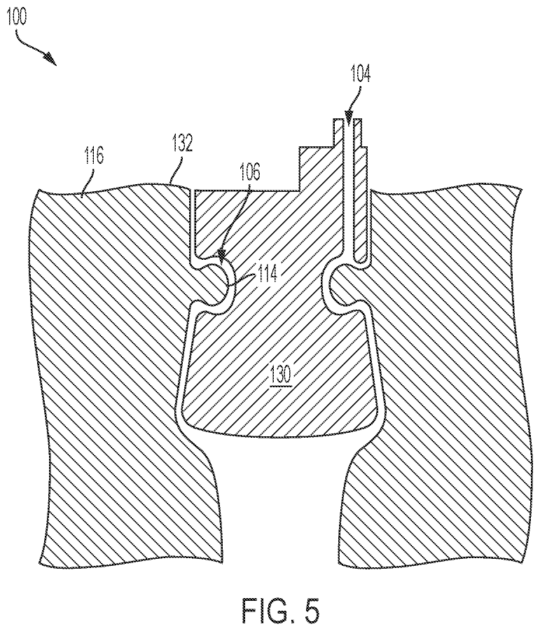

FIG. 5 provides a further embodiment of the disclosed technology. As shown, component 100 can be used to seal a wound in tissue 116. In the embodiment shown in FIG. 5, vacuum applied via tap 104 draws tented tissue 114 into vacuum chamber 106, which vacuum chamber can reside on or in projection 130: vacuum chamber 106 can be circumferential in configuration. As shown in FIG. 5, a component 100 can be configured to draw tissue into vacuum chamber 106 in a direction that is radially inward into the vacuum chamber 106. As shown in FIG. 5, component 100 can be at least partially inserted into a wound and then draws tissue circumferentially inward so as to seal the tissue wound, e.g., by sealing the component against the tissue.

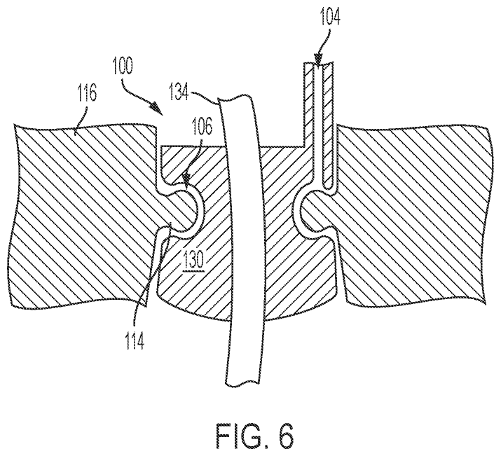

FIG. 6 provides a further embodiment of the disclosed technology. As shown, component 100 can be used to seal a wound in tissue 116. In the embodiment shown in FIG. 6, vacuum applied via tap 104 draws tented tissue 114 into vacuum chamber 106 (which can reside in or on projection 130): vacuum chamber 106 can be circumferential in configuration. As shown in FIG. 6, a component 100 can be configured to draw tissue into vacuum chamber 106 in a direction that is radially inward into the vacuum chamber 106. This can be contrasted with the embodiment of FIG. 1, which draws tissue upward rather than radially. As shown in FIG. 6, component 100 can be at least partially inserted into a wound and then draws tissue circumferentially inward so as to seal the tissue wound, e.g., by sealing the component against the tissue. Component 134 can also include conduit 134, which conduit can be used to deliver a medicament or other material through component 100 to an area within the body, e.g., an area that may have been wounded. Alternatively, conduit 134 could be used to withdraw fluid from a subject, e.g., fluid that has accumulated as a consequence of a wound.

### ASPECTS

The following Aspects are illustrative only and do not limit the scope of the present disclosure or the appended claims. Any part or parts of any one or more Aspects can be combined with any part or parts of any one or more other Aspects.

Aspect 1. A component, comprising: an upper surface, a bottom surface, a vacuum chamber defined between the upper surface and the lower surface, the bottom surface defining at least one vacuum chamber opening in fluid communication with the vacuum chamber, and the vacuum chamber opening being defined between vacuum chamber opening edges of the component: a vacuum tap that places the vacuum chamber into fluid communication with the environment exterior to the vacuum tap, the vacuum tap being adapted for application of vacuum within the vacuum chamber, the at least one vacuum chamber opening being adapted to, upon application of sufficient vacuum from the tap, securably engage with tissue proximate to the bottom surface by drawing at least some of the tissue toward (or even into) the vacuum chamber so as to stably affix the component to the tissue, and one or more of (i) a sealer portion, the sealer portion configured such that application of vacuum to draw tissue toward the vacuum chamber exerts the sealer against the tissue so as to seal an opening present in the tissue, (ii) a projection configured to extend into a wound when the component is engaged with a subject's tissue, the component optionally configured such that application of vacuum to draw tissue toward the vacuum chamber exerts the sealer against the tissue such that the projection extends into an opening of the tissue.

A component can comprise a rigid material, e.g., a metal, polycarbonate, and the like. A component can also comprise a flexible material, e.g., a resilient material that can be bent but then regains its shape. A component can also comprise a flexible material so as to allow a user to conform a surface of the component to a tissue (e.g., a blood vessel) that is being sealed.

A component can be configured (e.g., FIG. 1) such that when the component is resting atop a tissue surface, application of vacuum encourages tissue away from the surface and into the vacuum chamber. As shown in FIG. 5 and FIG. 6, a component can also be configured such that application of vacuum draws tissue radially inward toward the center of the component. As shown in FIG. 5 and FIG. 6, the tissue can be drawn radially inward into a vacuum chamber (106) that resides in or on a projection (130), which projection is configured to extend into a wound of the subject, e.g., a puncture or other wound.

As shown in FIG. 6, a component can comprise a conduit (e.g., a tube) that can be used to communicated fluid through the component to a subject and/or withdraw fluid from a subject. This allows a user to seal a wound as well as provide treatment and/or withdraw fluid from or nearby to a wound site. As but one example, medications, saline, or other fluids can be communicated through the tube to a subject. Instruments (e.g., cameras, cutting devices, and the like) can also be communicated through the tube into a subject and also withdrawn. Example vacuums are described in U.S. Pat. No. 9,277,884, the entirety of which patent is incorporated herein by reference for any and all purposes.

Aspect 2. The component of Aspect 1, the component defining an inner wall and a component opening at least partially bounded by the inner wall, the sealer portion comprising a member affixed so as to seal the component opening. Such a member can be a membrane, which can be present in the manner of the skin of a tambourine.

Aspect 3. The component of Aspect 2, wherein the member is a resilient member.

Aspect 4. The component of Aspect 2, wherein the member is a rigid member.

Aspect 5. The component of Aspect 2, wherein the member defines an opening therethrough, the opening being configured to communicate a material to a tissue contacted by the member.

Aspect 6. The component of Aspect 2, wherein the member is flush relative to the bottom surface of the component.

Aspect 7. The component of any one of Aspects 1-6, wherein the sealer portion comprises a medicament. Such medicaments can be, e.g., clotting factors, analgesics, and the like.

Aspect 8. The component of Aspect 7, wherein the medicament comprises a clotting promoter.

Aspect 9. The component of any one of Aspects 1-8, wherein a vacuum chamber edge of the component defines a thickened end.

Aspect 10. The component of any one of Aspects 1-9, comprising a plurality of vacuum chamber openings. As an example, a component can include a circular arrangement of openings, with application of vacuum to the openings affixing the component the tissue of interest. Vacuum chamber openings can be linear in shape (e.g., slots), but can also be of other shapes. As an example, a component can include four slots arranged in a square, rectangular, rhombus, or even trapezoidal shape. A component can also include two C-shaped slots arranged opposite one another to form a circle-like opening that faces the tissue of interest.

Aspect 11. The component of any one of Aspects 1-10, comprising a plurality of vacuum chambers defined within the component. A component can be arranged such that a single vacuum chamber is in fluid communication with more than one vacuum chamber opening. A component can also be arranged such that multiple vacuum chambers are in fluid communication with the same vacuum chamber opening.

Aspect 12. The component of any one of Aspects 1-11, further comprising a vacuum source in fluid communication with the vacuum tap. A vacuum source can be a motorized pump. A vacuum source can also be manually-operable, e.g., a squeeze bulb or other modality, such as a squeeze bulb having a one-way valve. A pressure sensor and/or gauge can be present in fluid communication with the vacuum chamber so as to allow the user to monitor the pressure within the vacuum chamber. A device can, for example, be configured to modulate the pressure within the vacuum chamber in an automated fashion, e.g., to modulate the pressure so as to maintain the pressure within the vacuum chamber within a certain range or ranges.

Aspect 13. The component of Aspect 12, wherein the vacuum source is manually operable to effect a vacuum. The vacuum source can also be automated, in addition to being manually operable.

Aspect 14. The component of Aspect 12, wherein the vacuum source is automated. The vacuum source can also be manually operable, in addition to being automated.

Aspect 15. The component of any one of Aspects 1-14, wherein the component defines a maximum cross-sectional dimension in the range of from about 0.5 to about 10 cm, e.g., from about 0.5 to about 10 cm, from about 1 to about 9 cm, from about 2 to about 8 cm, from about 3 to about 7 cm, from about 4 to about 6 cm, or even about 5 cm.

Aspect 16. The component of any one of Aspects 1-15, wherein the sealer portion defines a cross-sectional dimension in the range of from about 0.1 to about 5 cm, e.g., from about 0.1 to about 5 cm, from about 0.3 to about 4.7 cm, from about 0.5 to about 4.5 cm, from about 0.7 to about 4.1 cm, from about 1 to about 3.8 cm, from about 1.3 to about 3.3 cm, from about 1.7 to about 3 cm, from about 1.9 to about 2.7 cm, from about 2.2 to about 2.5 cm, or even about 2.4 cm.

Aspect 17. A method, comprising applying a vacuum via the vacuum tap of a device according to any one of Aspects 1-16 so as to sealably affix the component to tissue proximate to the component. As described elsewhere herein, the vacuum can be applied in an automated fashion (e.g., via an automated pump), but can also be applied in a manual fashion, e.g., via a squeeze bulb, straw, pedal, or other manual modality. A component can include a valve that is configured to limit the amount of vacuum applied by the component to tissue (e.g., limiting the applied vacuum to a level at or below a certain threshold); the amount of vacuum can also be modulated by the vacuum source.

Aspect 18. The method of Aspect 17, wherein applying the vacuum draws at least some of the tissue into the vacuum chamber so as to stably affix the component to the tissue.

Aspect 19. A kit, comprising a first component according to any one of Aspects 1-16 and a second component according to any one of Aspects 1-16, the first component and the second component defining different maximum cross-sectional dimensions. As but one example, a kit can comprise components of various sizes such that the kit allows a user to address wounds of varying size. Such a kit could be, for instance, be present in an emergency response vehicle so as to allow the emergency response personnel to address wounds of multiple sizes. The components of such a kit could be, of course, compatible with the same vacuum source so that a single vacuum source could be used to effect reduced pressure within multiple components at the same time.

Aspect 20. The kit of Aspect 19, wherein the sealer portion of first component and sealer portion of the second component define different cross-sectional dimensions. The first and second components can comprise projection portions, and the projection of the first component can differ in cross-sectional dimension (dimension W1 in FIG. 4) from the cross-sectional dimension of the projection of the second component. Likewise, the projection of the first component can differ in depth (dimension H1 in FIG. 4) from the depth of the projection of the second component.

1. A component, comprising:
an upper surface,
a bottom surface,
a vacuum chamber defined between the upper surface and the lower surface,
the bottom surface defining at least one vacuum chamber opening in fluid communication with the vacuum chamber, and
the vacuum chamber opening being defined between vacuum chamber opening edges of the component;
a vacuum tap that places the vacuum chamber into fluid communication with the environment exterior to the vacuum tap,
the vacuum tap being adapted for application of vacuum within the vacuum chamber,
the at least one vacuum chamber opening being adapted to, upon application of sufficient vacuum from the tap, securably engage with tissue proximate to the bottom surface by drawing at least some of the tissue toward (or even into) the vacuum chamber so as to stably affix the component to the tissue, and
one or more of
(i) a sealer portion, the sealer portion configured such that application of vacuum to draw tissue toward the vacuum chamber exerts the sealer against the tissue so as to seal an opening present in the tissue,
(ii) a projection configured to extend into a wound when the component is engaged with a subject's tissue, the component optionally configured such that application of vacuum to draw tissue toward the vacuum chamber exerts the sealer against the tissue such that the projection extends into an opening of the tissue.

2. The component of claim 1, the component defining an inner wall and a component opening at least partially bounded by the inner wall, the sealer portion comprising a member affixed so as to seal the component opening.

3. The component of claim 2, wherein the member is a resilient member.

4. The component of claim 2, wherein the member is a rigid member.

5. The component of claim 2, wherein the member defines an opening therethrough, the opening being configured to communicate a material to a tissue contacted by the member.

6. The component of claim 2, wherein the member is flush relative to the bottom surface of the component.

7. The component of claim 1, wherein the sealer portion comprises a medicament.

8. The component of claim 6, wherein the medicament comprises a clotting promoter.

9. The component of claim 1, wherein a vacuum chamber edge of the component defines a thickened end.

10. The component of claim 1, comprising a plurality of vacuum chamber openings.

11. The component of claim 1, comprising a plurality of vacuum chambers defined within the component.

12. The component of claim 1, further comprising a vacuum source in fluid communication with the vacuum tap.

13. The component of claim 12, wherein the vacuum source is manually operable to effect a vacuum.

14. The component of claim 12, wherein the vacuum source is automated.

15. The component of claim 1, wherein the component defines a maximum cross-sectional dimension in the range of from about 0.05 to about 10 cm.

16. The component of claim 1, wherein the sealer portion defines a cross-sectional dimension in the range of from about 0.05 to about 10 cm.

17. A method, comprising applying a vacuum via the vacuum tap of a device according to claim 1 so as to sealably affix the component to tissue proximate to the component.

18. The method of claim 17, wherein applying the vacuum draws at least some of the tissue into the vacuum chamber so as to stably affix the component to the tissue.

19. A kit, comprising a first component according to claim 1 and a second component according to claim 1, the first component and the second component defining different maximum cross-sectional dimensions.

20. The kit of claim 19, wherein the sealer portion of first component and sealer portion of the second component define different cross-sectional dimensions.
